(19) United States
(12) Patent Application Publication
(10) Pub. No.: US 2025/0376479 A1
(43) Pub. Date: Dec. 11, 2025

(54) EXATECAN DERIVATIVES AND USES THEREOF

(71) Applicant: PRIMELINK BIOTHERAPEUTICS (SUZHOU) CO., LTD. LTD., Suzhou (CN)

(72) Inventors: Yingdong LU, Suzhou (CN); Guangchao ZHANG, Suzhou (CN); Xiaohe SHI, Suzhou (CN); Yu SHEN, Suzhou (CN); Jiani YU, Suzhou (CN); Mao YIN, Needham, MA (US)

(21) Appl. No.: 19/231,579

(22) Filed: Jun. 9, 2025

(30) Foreign Application Priority Data
Jun. 11, 2024 (WO) PCT/CN2024/098437

(51) Int. Cl. C07D 491/22; A61K 31/4745; A61P 35/00

(52) U.S. Cl. CPC C07D 491/22; A61K 31/4745; A61P 35/00

(57) ABSTRACT

Disclosed herein are compounds or pharmaceutically acceptable salts thereof that are useful as topoisomerase I (Topo I) inhibitors. Also disclosed are pharmaceutical compositions comprising such compounds, and methods of using such compounds or compositions to treat diseases, such as cancers.

### FIELD OF THE DISCLOSURE

The present disclosure generally relates to compounds or pharmaceutically acceptable salts thereof that may be useful as topoisomerase I (Topo I) inhibitors. The present disclosure also relates to the synthesis process of such compounds and the use of such compounds for the treatment of diseases, such as cancers.

### BACKGROUND

Topoisomerase I is an essential enzyme involved in the regulation of DNA supercoiling during transcription and replication processes. The discovery of Topo I inhibitors has been a significant milestone in the development of anticancer drugs, as these compounds exploit the enzyme's function to induce DNA damage and cell death in cancer cells.

The first Topo I inhibitor to be approved for clinical use was camptothecin, a natural product derived from the Chinese tree Camptotheca acuminata. However, the clinical application of camptothecin was limited due to its poor solubility and instability. Exatecan is a water-soluble camptothecin derivative. As a chemotherapeutic agent, exatecan mesylate did not gain drug approval after several clinical trials due to lack of efficacy or high toxicity at tested doses.

Recently, the development of antibody-drug conjugates (ADCs) for delivery of Topo I inhibitors to diseased tissues could lead to improved safety and efficacy.

Thus, there is a need for new compounds with proper potency and property such as membrane permeability, which provide good bystander effects.

### SUMMARY OF THE INVENTION

The present disclosure includes compounds of Formula (I) or pharmaceutically acceptable salts thereof. These compounds enable the inhibition of Topo I to treat various diseases, including but not limited to cancer.

In one aspect, the present disclosure provides a compound of Formula (I).

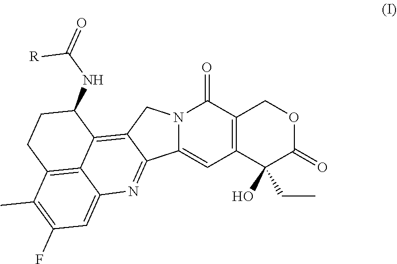

or a pharmaceutically acceptable salt thereof, wherein:
R is selected from the group consisting of alkyl, alkoxy, heteroalkyl, cycloalkyl, and heterocyclyl, each optionally substituted with one or more R^{L};
R^{L} in each occurrence is independently selected from halogen, hydroxyl, cyano, alkyl, alkoxy, haloalkyl, —OR^{a} or —N(R^{b})(R^{c});
each of R^{a}, R^{b} and R^{c} in each occurrence is independently hydrogen or alkyl.

In another aspect, the present disclosure provides a compound or a pharmaceutically acceptable salt thereof, wherein the compound is selected from the group consisting of:

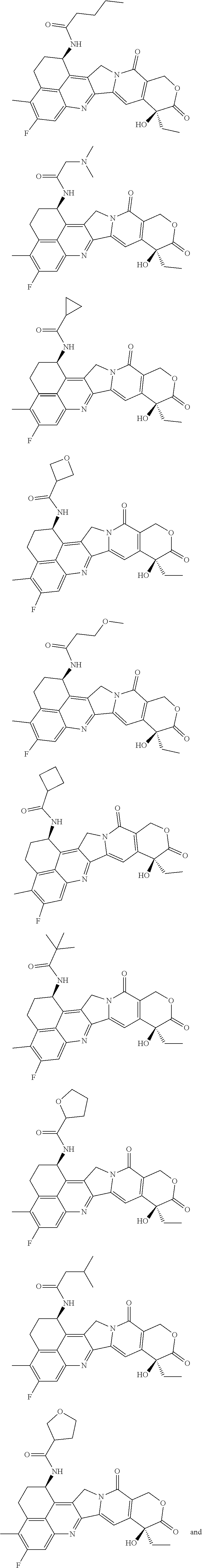

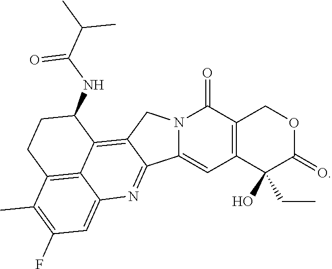 [SMILES: CC[C@@]1(O)C(=O)OCc2c1cc1n(c2=O)Cc2c-1nc1cc(F)c(C)c3c1c2[C@H](NC(=O)C(C)C)CC3]

In another aspect, the present disclosure provides a pharmaceutical composition comprising the compound of the present disclosure or a pharmaceutically acceptable salt thereof, and a pharmaceutically acceptable carrier.

In a further aspect, the present disclosure provides a method of treating a cancer in a subject in need thereof comprising administering to the subject an effective amount of the compound of the present disclosure or a pharmaceutically acceptable salt thereof or the pharmaceutical composition of the present disclosure.

In another aspect, the present disclosure provides use of the compound of the present disclosure or a pharmaceutically acceptable salt thereof or the pharmaceutical composition of the present disclosure in the manufacture of a medicament for treating cancers.

In a further aspect, the present disclosure provides a compound of present disclosure or a pharmaceutically acceptable salt thereof or the pharmaceutical composition of the present disclosure, for use in the treatment of cancers.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, —NR(CR′R″)— includes both —NR(CR′R″)— and —(CR′R″)NR—. Where the structure clearly requires a linking group, the Markush variables listed for that group are understood to be linking groups. if the structure requires a linking group and the Markush group definition for that variable lists “alkyl”, then it is understood that the “alkyl” represents a linking alkylene group.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, a dash “-” at the front or end of a chemical group is used, a matter of convenience, to indicate a point of attachment for a substituent. For example, —OH is attached through the oxygen atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “n is an integer between 1 and 6” and “n being an integer of 1 to 6” both mean “n being 1, 2, 3, 4, 5, or 6”.

As used herein, the term “compounds provided herein”, or “compounds disclosed herein” or “compounds of the present disclosure” refers to the compounds of Formula (I) as well as the specific compounds disclosed herein.

As used herein, the term “Cᵢ₋ⱼ” indicates a range of the carbon atoms numbers, wherein i and j are integers, and the range of the carbon atoms numbers includes the endpoints (i.e., i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkoxy”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxy” include, but are not limited to, methoxy, ethoxy, propoxy (e.g. n-propoxy and isopropoxy), t-butoxy, neopentoxy, n-hexoxy, and the like.

As used herein, the term “cyano” refers to —CN.

As used herein, the term “cycloalkyl”, whether as part of another term or used independently, refers to a non-aromatic, saturated or partially unsaturated monocyclic or polycyclic ring system optionally substituted independently with one or more substituents described below, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. In the case of polycyclic ring system, the fused, spiro and bridged ring systems are included within the scope of this definition. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “halogen” refers to an atom selected from fluorine (or fluoro), chlorine (or chloro), bromine (or bromo) and iodine (or iodo).

As used herein, the term “haloalkyl”, whether as part of another term or used independently, refers to an alkyl group having one or more halogen substituents. Examples of haloalkyl group include, but are not limited to, trifluoromethyl (—CF₃), pentafluoroethyl (—C₂F₅), difluoromethyl (—CHF₂), trichloromethyl (—CCl₃), dichloromethyl (—CHCl₂), pentachloroethyl (—C₂Cl₅), and the like.

As used herein, the term “heteroatom” refers to nitrogen, oxygen, sulfur, phosphorus, and includes any oxidized form of nitrogen, sulfur or phosphorus, and any quaternized form of a basic nitrogen (including N-oxides).

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxy and heteroalkoxy radicals.

As used herein, the term “heterocyclyl” refers to a saturated or partially unsaturated cycloalkyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen, sulfur or phoshporus, and any quaternized form of a basic nitrogen. “Heterocyclyl” also includes radicals wherein the heterocyclyl radicals are fused with a saturated or partially unsaturated carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

As used herein, the term “hydroxyl” or “hydroxy” refers to —OH.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “one or more” item includes a single item selected from the list as well as mixtures of two or more items from the list.

As used herein, the term “optional” or “optionally” means that the subsequently described event or circumstance may or may not occur, and that the description includes instances where the said event or circumstance occurs and instances in which it does not. As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compound

In one aspect, the present disclosure provides compounds of Formula (I):

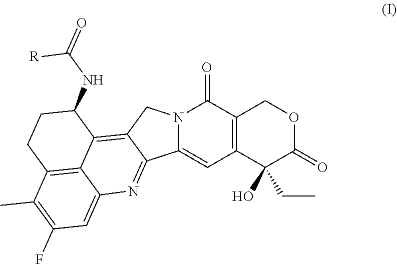

or a pharmaceutically acceptable salt thereof, wherein:
R is selected from the group consisting of alkyl, alkoxy, heteroalkyl, cycloalkyl, and heterocyclyl, each optionally substituted with one or more R^{L};
R^{L} in each occurrence is independently selected from halogen, hydroxyl, cyano, alkyl, alkoxy, haloalkyl, —OR^{a} or —N(R^{b})(R^{c});
each of R^{a}, R^{b} and R^{c} in each occurrence is independently hydrogen or alkyl.

In some embodiments, R is selected from the group consisting of C₁₋₆alkyl, C₁₋₆alkoxy, C₁₋₆heteroalkyl, C₃₋₁₀cycloalkyl, and 3- to 12-membered heterocyclyl, each of which is optionally substituted with one or more R^{L}.

In some embodiments, R is C₁₋₆alkyl. In certain embodiments, R is methyl, ethyl, propyl, butyl, pentyl or hexyl.

In some embodiments, R is C₁₋₆heteroalkyl. In certain embodiments, R is C₁₋₆heteroalkyl containing one or more heteroatoms selected from N, O or S. In certain embodiments, R is —CH₂N(CH₃)₂ or —CH₂CH₂OCH₃.

In some embodiments, R is C₃₋₁₀cycloalkyl. In certain embodiments, R is cyclopropyl or cyclobutyl.

In some embodiments, R is 3- to 12-membered heterocyclyl. In certain embodiments, R is 3- to 12-membered heterocyclyl containing one or more heteroatoms selected from N, O or S. In certain embodiments, R is

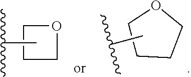

In some embodiments, R^{L} in each occurrence is independently selected from —OR^{a} or —N(R^{b})(R^{c}), and each of R^{a}, R^{b} and R^{c} in each occurrence is independently C₁₋₆alkyl.

Exemplary compounds of the present disclosure are set forth below.

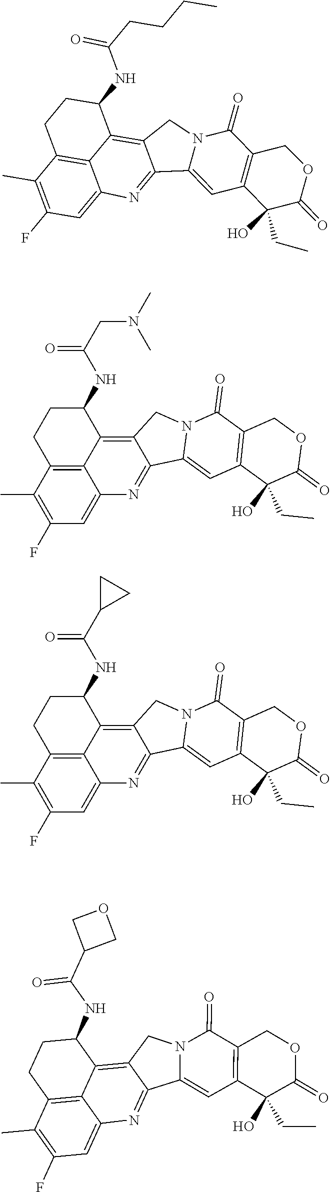 [SMILES: CCCCC(=O)N[C@@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@H](NC(=O)CN(C)C)CC3 ; CC[C@@]1(O)C(=O)OCc2c1cc1n(c2=O)Cc2c-1nc1cc(F)c(C)c3c1c2[C@H](NC(=O)C1CC1)CC3 ; CC[C@@]1(O)C(=O)OCc2c1cc1n(c2=O)Cc2c-1nc1cc(F)c(C)c3c1c2[C@H](NC(=O)C1COC1)CC3]

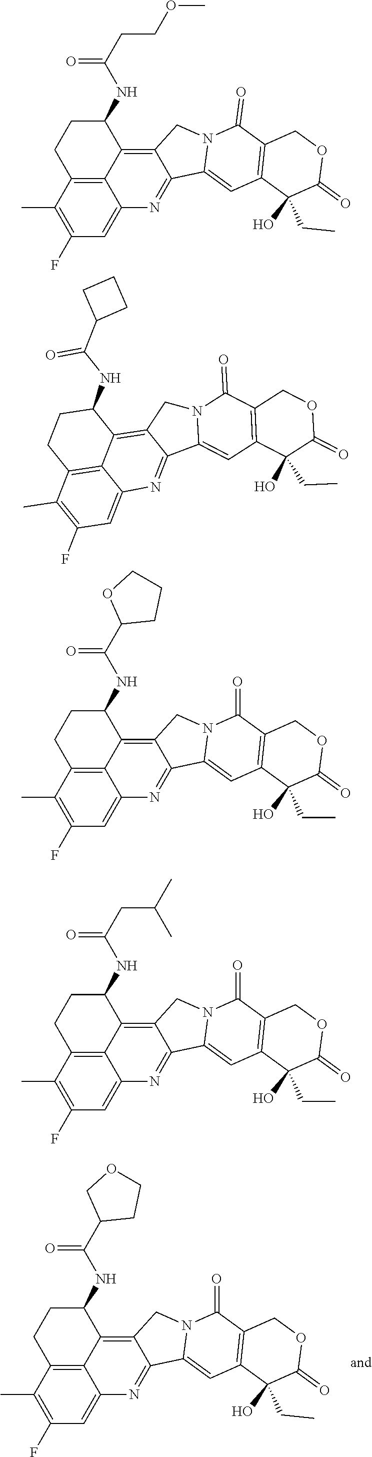

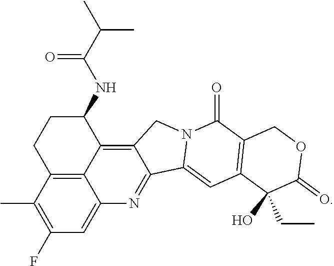 [SMILES: CC[C@@]1(O)C(=O)OCc2c1cc1n(c2=O)Cc2c-1nc1cc(F)c(C)c3c1c2[C@H](NC(=O)C(C)C)CC3]

Compounds provided herein are described with reference to both generic formulae and specific compounds. In addition, the compounds of the present disclosure may exist in a number of different forms or derivatives, including but not limited to prodrugs, active metabolic derivatives (active metabolites), solvates, pharmaceutically acceptable salts or isotope derivatives, all of which are within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

In the compounds or pharmaceutically acceptable salts thereof provided herein, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is intended to include all isotopic variations of the compounds provided herein. For example, different isotopic forms of hydrogen include protium (¹H), deuterium (²H) and tritium (³H), and different isotopic forms of carbon includes ¹²C and ¹³C. Unless otherwise specified, hydrogen, carbon, nitrogen, oxygen, phosphorous, sulfur, fluorine, chlorine, bromide or iodine in the compounds or pharmaceutically acceptable salts thereof of present disclosure are meant to include their isotopic forms, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I.

Compounds provided herein or pharmaceutically acceptable salts thereof may contain one or more asymmetric centers and thus give rise to enantiomers, diastereomers, and other stereoisomeric forms that may be defined, in terms of absolute stereochemistry, as (R)- or (S)- or, as (D)- or (L)- for amino acids, or in terms of relative configuration, as rel-(R)— or rel-(S)—. The present disclosure includes all such possible isomers, as well as their racemic and optically pure forms. Optically active (+) and (−), (R)- and(S)-, or (D)- and (L)-isomers may be prepared using chiral synthons or chiral reagents, or resolved by conventional techniques, such as, chromatography and fractional crystallization. Traditional techniques for the preparation, isolation of individual enantiomers include chiral synthesis from a suitable optically pure precursor or resolution of the racemate (or the racemate of a salt or derivative) using, for example, chiral high pressure liquid chromatography (HPLC). Wherever compounds are represented in their chiral form, it is understood that the embodiment includes, but is not limited to, the specific diastereomerically or enantiomerically enriched form. In situations that the chirality is not specified but is present, it is understood that the embodiment is intended to include either the specific diastereomerically or enantiomerically enriched form; or a racemic or scalemic mixture of such compound(s).

When the compounds provided herein contain olefinic double bonds or other centers of geometric asymmetry, and unless specified otherwise, these compounds include both E and Z geometric isomers.

### Synthetic Method

In some embodiments, the present disclosure provides a method of preparing the compounds provided herein.

In some embodiments, the present disclosure provides a method of a compound provided herein, comprising one or more steps as described herein.

In some embodiments, the present disclosure provides a compound obtainable by, or obtained by a method for preparing a compound as described herein.

In some embodiments, the present disclosure provides an intermediate as described herein, which is suitable for use in a method for preparing a compound as described herein.

The compounds provided herein can be prepared using any suitable organic synthesis techniques and can be synthesized according to any of numerous possible synthetic routes. Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

By way of example, a suitable protecting group for an amino or alkylamino group is, for example, an acyl group, an alkanoyl group, an alkoxycarbonyl group, an arylmethoxycarbonyl group, or an aroyl group. A suitable protecting group for a hydroxy group is, for example, an acyl group, an alkanoyl group an aroyl group, or an arylmethyl group. A suitable protecting group for a carboxy group is, for example, an esterifying group, for example a methyl, an ethyl group, or a benzyl group.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6 (6), 874-883, which is incorporated herein by reference in its entirety), and normal phase silica chromatography.

Several methods for preparing the compounds of Formula (I), and pharmaceutically acceptable salts thereof, are described in, for example, the following examples. Starting materials and intermediates are purchased from commercial sources, made from known procedures, or are otherwise illustrated. In some cases, the order of carrying out the steps of the reaction schemes may be varied to facilitate the reaction or to avoid unwanted reaction products.

### Pharmaceutical Composition

In a further aspect, there is provided pharmaceutical compositions comprising one or more compounds of the present disclosure, or a pharmaceutically acceptable salt thereof. In some embodiments, the pharmaceutical compositions of the present disclosure comprise a first compound provided herein or a pharmaceutically acceptable salt thereof and one or more additional compounds of the same formula but said first compound and additional compounds are not the same molecules.

In another aspect, there is provided pharmaceutical composition comprising one or more compounds of the present disclosure, or a pharmaceutically acceptable salt thereof, and at least one pharmaceutical acceptable excipient.

In some embodiments, the pharmaceutical composition of the present disclosure comprises a therapeutically effective amount of one or more compounds of the present disclosure or a pharmaceutically acceptable salt thereof.

In some embodiments, the pharmaceutical composition of the present disclosure comprises a therapeutically effective amount of one or more compounds of the present disclosure or a pharmaceutically acceptable salt thereof, and at least one pharmaceutical acceptable excipient.

As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

As used herein, the term “pharmaceutical composition” refers to a formulation containing the molecules or compounds of the present disclosure in a form suitable for administration to a subject. The pharmaceutical compositions include compositions suitable adapted for oral administration, rectal administration, topical administration, parenteral (including subcutaneous, intramuscular, and intravenous) administration, sublingual administration, ocular administration, transdermal administration or nasal administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure are being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

In certain embodiments, the pharmaceutical compositions of the present disclosure may be in solid dosage forms for oral administration (capsules, tablets, pills, dragees, powders, granules and the like). Suitable pharmaceutically acceptable excipients include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and antioxidants, such as ascorbic acid.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, antioxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an antioxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally occurring gums such as gum acacia or gum tragacanth, naturally occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

The dosage regimen for the compounds provided herein will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disorder.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as a single dosage form. The amount of the compounds provided herein in the single dosage form will vary depending on the subject treated and particular mode of administration. In some embodiments, dosage forms suitable for administration may contain from about 1 mg to about 1000 mg of active ingredient per dosage unit. In these pharmaceutical compositions the active ingredient will ordinarily be present in an amount of about 0.1-95% by weight based on the total weight of the composition.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In some embodiments, a dose of the compounds provided herein or the pharmaceutical compositions provided herein is administered to a subject every day, every other day, every couple of days, every third day, once a week, twice a week, three times a week, or once every two weeks. If desired, the effective daily dose of the active compound may be administered as two, three, four, five, six or more sub-doses administered separately at appropriate intervals throughout the day, optionally, in unit dosage forms. In some embodiments, a dose of the compounds provided herein or the pharmaceutical compositions provided herein is administered for 2 days, 3 days, 5 days, 7 days, 14 days, 21 days, 1 month, 2 months, 2.5 months, 3 months, 4 months, 5 months, 6 months or more.

### Combinations

In some embodiments, the compounds provided herein, or pharmaceutically acceptable salts thereof may be administered in combination with one or more additional therapeutically active agents. The additional therapeutically active agents may have complementary activities to the compounds provided herein or pharmaceutically acceptable salts thereof such that they do not adversely affect each other. Such agents are suitably present in combination in amounts that are effective for the purpose intended. In some embodiments, such agents are those previously been shown to treat cancers.

The additional therapeutic agent or agents may be administered simultaneously or sequentially with the compounds provided herein. Sequential administration includes administration before or after the compounds provided herein. In some embodiments, the additional therapeutic agent or agents may be administered in the same composition as the compounds provided herein. In other embodiments, there may be an interval of time between administration of the additional therapeutic agent and the compounds provided herein.

In some embodiments, the administration of an additional therapeutic agent with a compound provided herein may enable lower doses of the other therapeutic agents and/or administration at less frequent intervals.

### Method for Treatment

Compounds of the present disclosure and pharmaceutical composition comprising the same function as Topo I inhibitors for preventing or treating cancers.

In a further aspect, the present disclosure provides a method of treating cancers, comprising administering an effective amount of the compound or a pharmaceutically acceptable salt thereof or the pharmaceutical composition provided herein to a subject in need thereof.

As used herein, the term “treating”, “treatment” or “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treating” can also mean prolonging survival as compared to expected survival if not receiving it. Those in need of therapy include those already with the condition or disorder as well as those prone to have the condition or disorder or those in which the condition or disorder is to be prevented.

As used herein, the term “preventing”, “prevention” or “prophylaxis” is intended to have its normal meaning and includes primary prophylaxis to prevent the development of the disease and secondary prophylaxis whereby the disease has already developed, and the patient is temporarily or permanently protected against exacerbation or worsening of the disease, or the development of new symptoms associated with the disease.

In some embodiments, the compounds or pharmaceutically acceptable salts thereof and the compositions provided herein may be used for the treatment cancer, including but not limited to lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer. In some embodiments, the cancer is present in an adult patient. In some embodiments, the cancer is present in a pediatric patient.

The concentration and route of administration to the subject will vary depending on the cancers to be treated. In certain embodiments, the administration is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and ocular administration.

The compounds of the present disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted herein, a compound of the present disclosure may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. A suitable treatment period can be, for example, at least about one week, at least about two weeks, at least about one month, at least about six months, at least about 1 year, or indefinitely. A treatment period can terminate when a desired result is achieved.

### EXAMPLES

The followings further explain the general methods of the present disclosure. The compounds of the present disclosure may be prepared by the methods known in the art. The following illustrates the detailed preparation methods of the preferred compounds of the present disclosure. However, they are by no means limiting the preparation methods of the compounds of the present disclosure.

### Synthetic Examples

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

### Example 1 Synthesis of Compound 1

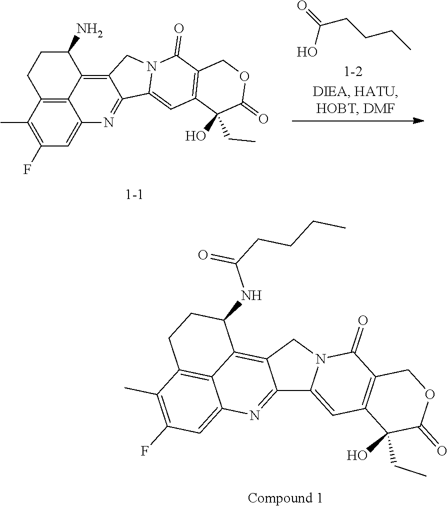

To a solution of Compound 1-1 (50.0 mg, 0.11 mmol) in DMF (1.0 mL) was added Compound 1-2 (29.7 mg, 0.33 mmol), HOBT (22.3 mg, 0.17 mmol) and HATU (62.7 mg, 0.17 mmol). The mixture was stirred at 0° C. for 10 min. DIEA (42.6 mg, 0.33 mmol) was added to the mixture and stirred at 20° C. for 16 hours. The mixture reaction was purified by C18 column chromatography eluted with ACN/H₂O (0.1% FA) (20%˜55%) to give Compound 1 (35.2 mg, Yield 60.06%).

LCMS (5%-95%, 1 cm, Rt=10.024 min): [M+H]+520.0

¹H NMR (400 MHZ, DMSO-d6) δ 8.42 (d, J=8.4 Hz, 1H), 7.78 (d, J=10.8 Hz, 1H), 7.30 (s, 1H), 6.51 (s, 1H), 5.59-5.52 (m, 1H), 5.42 (s, 2H), 5.17 (q, J=18.8 Hz, 2H), 3.16 (s, 2H), 2.39 (s, 3H), 2.20-2.09 (m, 4H), 1.92-1.80 (m, 2H), 1.61-1.52 (m, 2H), 1.35-1.25 (m, 2H), 0.90-0.84 (m, 6H).

### Example 2 Synthesis of Compound 2

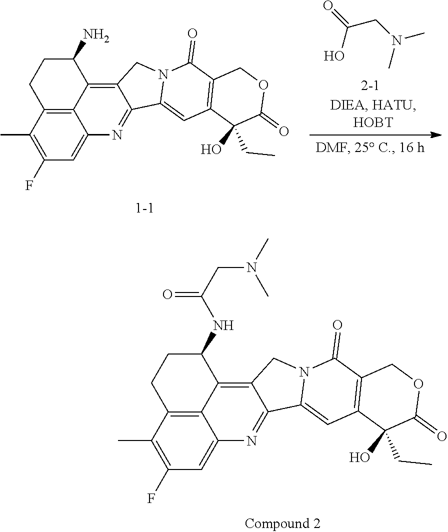

To a solution of Compound 1-1 (50.0 mg, 0.11 mmol) in DMF (2.0 mL) was added Compound 2-1 (17.0 mg, 0.17 mmol), HOBT (22.3 mg, 0.17 mmol) and HATU (62.7 mg, 0.17 mmol). The mixture was stirred at 0° C. for 10 min. DIEA (0.055 mL, 0.33 mmol) was added to the mixture and stirred at 25° C. for 16 hours. The mixture reaction was purified by C18 column chromatography eluted with ACN/H₂O (0.1% FA) (20%˜30%) to give Compound 2 (10.3 mg, Yield 16.68%).

LCMS (5%-95%, 1 cm, Rt=1.042 min): [M+H]+521.0

¹H NMR (400 MHZ, DMSO-d6) δ 8.46 (d, J=8.8 Hz, 1H) 7.79 (d, J=11.2 Hz, 1H), 7.31 (s, 1H), 6.51 (s, 1H), 5.62-5.48 (m, 1H), 5.42 (s, 2H), 5.20 (s, 2H), 3.19-3.13 (m, 2H), 3.00 (d, J=11.6 Hz, 2H), 2.40 (s, 3H), 2.24 (s, 6H), 2.18-2.14 (m, 2H), 1.92-1.78 (m, 2H), 0.87 (t, J=7.2 Hz, 3H).

### Example 3 Synthesis of Compound 3

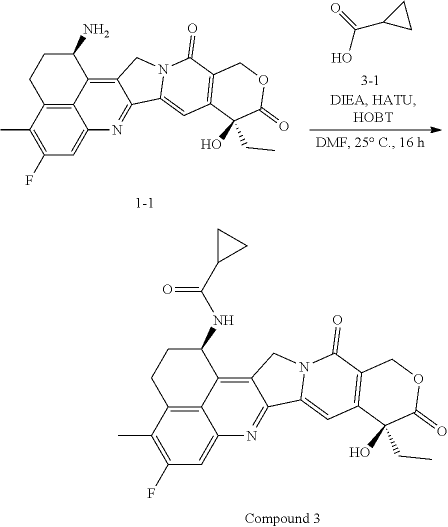 [SMILES: CC[C@@]1(O)C(=O)OCc2c1cc1n(c2=O)Cc2c-1nc1cc(F)c(C)c3c1c2[C@H](N)CC3 ; O=C(O)C1CC1 ; CC[C@@]1(O)C(=O)OCc2c1cc1n(c2=O)Cc2c-1nc1cc(F)c(C)c3c1c2[C@H](NC(=O)C1CC1)CC3]

To a solution of Compound 1-1 (50.0 mg, 0.11 mmol) in DMF (2.0 mL) was added Compound 3-1 (14.1 mg, 0.17 mmol), HOBT (22.3 mg, 0.17 mmol) and HATU (62.7 mg, 0.17 mmol). The mixture was stirred at 0° C. for 10 min. DIEA (0.055 mL, 0.33 mmol) was added to the mixture and stirred at 25° C. for 16 hours. The mixture reaction was purified by C18 column chromatography eluted with ACN/H₂O (0.1% FA) (20%˜50%) to give Compound 3 (23.2 mg, Yield 39.83%).

LCMS (5%-95%, 1 cm, Rt=1.476 min): [M+H]+504.1

¹H NMR (400 MHZ, DMSO-d6) δ 8.70 (d, J=8.8 Hz, 1H), 7.79 (d, J=10.8 Hz, 1H), 7.30 (s, 1H), 6.51 (s, 1H), 5.62-5.53 (m, 1H), 5.42 (s, 2H), 5.16 (s, 2H), 3.28-3.10 (m, 2H), 2.40 (s, 3H), 2.21-1.98 (m, 2H), 1.91-1.75 (m, 2H), 1.68-1.57 (m, 1H), 0.92-0.79 (m, 5H), 0.76-0.68 (m, 2H).

### Example 4 Synthesis of Compound 4

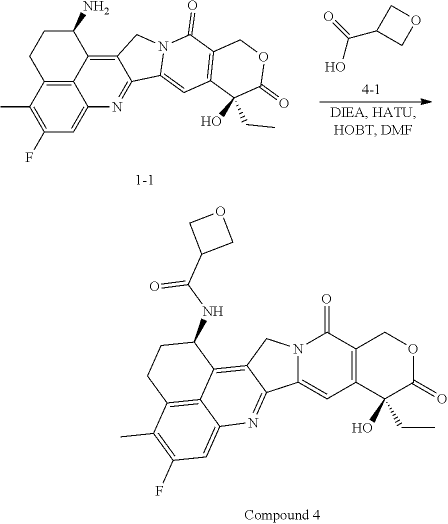 [SMILES: CC[C@@]1(O)C(=O)OCc2c1cc1n(c2=O)Cc2c-1nc1cc(F)c(C)c3c1c2[C@H](N)CC3 ; O=C(O)C1COC1 ; CC[C@@]1(O)C(=O)OCc2c1cc1n(c2=O)Cc2c-1nc1cc(F)c(C)c3c1c2[C@H](NC(=O)C1COC1)CC3]

To a solution of Compound 1-1 (50.0 mg, 0.11 mmol) in DMF (1.0 mL) was added Compound 4-1 (16.8 mg, 0.17 mmol), HOBT (22.3 mg, 0.17 mmol) and HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% FA) (35%˜55%) to give Compound 4 (40.3 mg, Yield 67.05%).

LCMS (5%-95%, 1 cm, Rt=1.357 min): [M+H]+520.1

¹H NMR (400 MHZ, DMSO-d6) δ 8.48 (d, J=8.8 Hz, 1H), 7.79 (d, J=11.2 Hz, 1H), 7.30 (s, 1H), 6.51 (s, 1H), 5.65-5.58 (m, 1H), 5.42 (s, 2H), 5.28-5.08 (m, 2H), 4.74-4.58 (m, 4H), 3.85-3.74 (m, 1H), 3.16 (s, 2H), 2.40 (s, 3H), 2.15 (s, 2H), 1.91-1.78 (m, 2H), 0.86 (t, J=7.2 Hz, 3H)

### Example 5 Synthesis of Compound 5

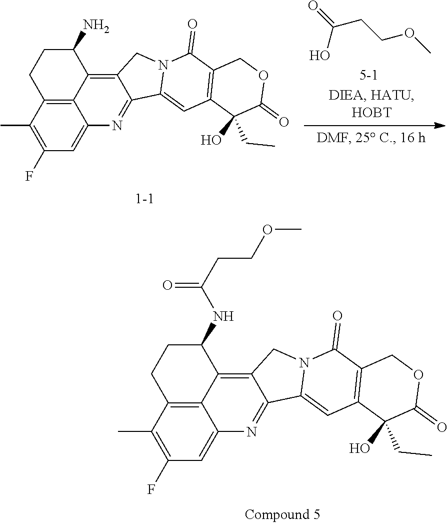

To a solution of Compound 1-1 (50.0 mg, 0.11 mmol) in DMF (2.0 mL) was added Compound 5-1 (11.0 mg, 0.11 mmol), HOBT (22.3 mg, 0.17 mmol) and HATU (62.7 mg, 0.17 mmol). The mixture was stirred at 0° C. for 10 min. DIEA (0.055 mL, 0.33 mmol) was added to the mixture and stirred at 25° C. for 16 hours.

The mixture reaction was purified by C18 column chromatography eluted with ACN/H₂O (0.1% FA) (20%˜45%) to give Compound 5 (23.7 mg, Yield 38.77%).

LCMS (5%-95%, 1 cm, Rt=1.411 min): [M+H]+522.0

¹H NMR (400 MHZ, DMSO-d6) δ 8.48 (d, J=8.8 Hz, 1H), 7.79 (d, J=11.2 Hz, 1H), 7.30 (s, 1H), 5.59-5.51 (m, 1H), 5.42 (s, 2H), 5.20 (d, J=5.2 Hz, 2H), 3.68-3.52 (m, 3H), 3.19-3.08 (m, 2H), 2.43-2.36 (m, 5H), 2.19-2.06 (m, 2H), 1.92-1.75 (m, 2H), 0.87 (t, J=7.2 Hz, 3H).

### Example 6 Synthesis of Compound 6

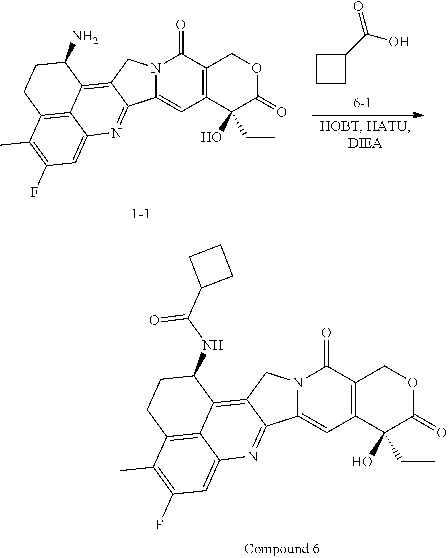 [SMILES: CC[C@@]1(O)C(=O)OCc2c1cc1n(c2=O)Cc2c-1nc1cc(F)c(C)c3c1c2[C@H](N)CC3 ; O=C(O)C1CCC1 ; CC[C@@]1(O)C(=O)OCc2c1cc1n(c2=O)Cc2c-1nc1cc(F)c(C)c3c1c2[C@H](NC(=O)C1CCC1)CC3]

To a solution of Compound 1-1 (50.0 mg, 0.11 mmol) in DMF (2.0 mL) was added Compound 6-1 (16.5 mg, 0.17 mmol), HOBT (22.3 mg, 0.17 mmol), HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% TFA) (44%-54%) to give Compound 6 (11.9 mg, Yield 20.65%).

LCMS (5%-95%, 1 cm, Rt=1.530 min): [M+H]+518.1

¹H NMR (400 MHZ, MeOD-d4) δ 7.57 (s, 1H), 7.54 (d, J=10.8 Hz, 1H), 5.65-5.59 (m, 1H), 5.54 (d, J=16.4 Hz, 1H), 5.33 (d, J=16.0 Hz, 1H), 5.12 (d, J=19.2 Hz, 1H), 4.93 (d, J=18.8 Hz, 1H), 3.24-3.16 (m, 3H), 2.46-2.40 (m, 2H), 2.38 (s, 3H), 2.33-2.22 (m, 5H), 2.06-2.01 (m, 1H), 1.96-1.90 (m, 3H), 0.99 (t, J=7.2 Hz, 3H).

### Example 7 Synthesis of Compound 7

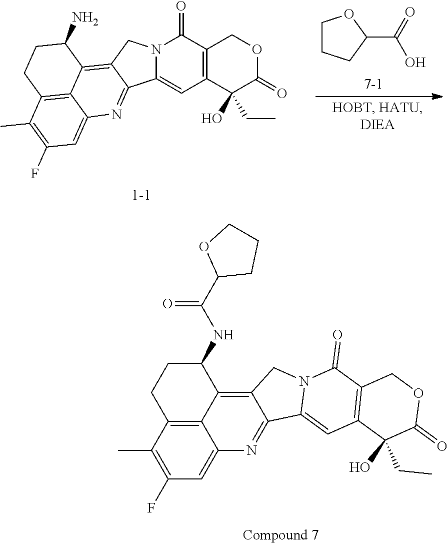 [SMILES: CC[C@@]1(O)C(=O)OCc2c1cc1n(c2=O)Cc2c-1nc1cc(F)c(C)c3c1c2[C@H](N)CC3 ; O=C(O)C1CCCO1 ; CC[C@@]1(O)C(=O)OCc2c1cc1n(c2=O)Cc2c-1nc1cc(F)c(C)c3c1c2[C@H](NC(=O)C1CCCO1)CC3]

To a solution of Compound 1-1 (50.0 mg, 0.11 mmol) in DMF (2.0 mL) was added Compound 7-1 (19.2 mg, 0.17 mmol), HOBT (22.3 mg, 0.17 mmol), HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% FA) (39%˜49%) to give Compound 7 (31.8 mg, Yield 40.65%).

LCMS (5%-95%, 1 cm, Rt=1.480 min): [M+H]+534.1

¹H NMR (400 MHZ, DMSO-d6) δ 8.60-8.51 (m, 1H), 7.80-7.73 (m, 1H), 7.30 (s, 1H), 5.54 (d, J=8.0 Hz, 1H), 5.57-5.53 (m, 2H), 5.22-5.10 (m, 2H), 4.39-4.33 (m, 1H), 4.02-3.88 (m, 1H), 3.81-3.73 (m, 1H), 3.22-3.13 (m, 2H), 2.39 (s, 3H), 2.28-2.08 (m, 4H), 2.07-1.79 (m, 5H), 0.87 (t, J=7.2 Hz, 3H).

### Example 8 Synthesis of Compound 8

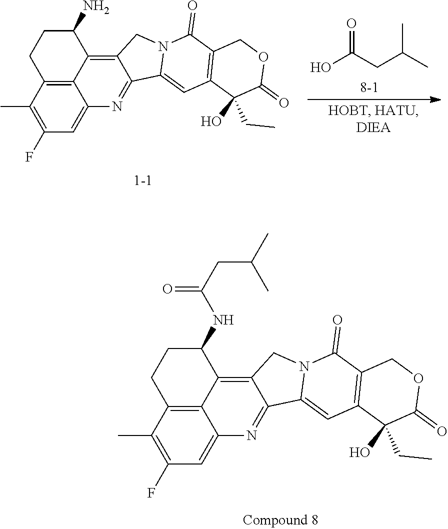

To a solution of Compound 1-1 (50.0 mg, 0.11 mmol) in DMF (2.0 mL) was added Compound 8-1 (17.2 mg, 0.17 mmol), HOBT (22.3 mg, 0.17 mmol), HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% FA) (45%˜55%) to give to give Compound 8 (35.3 mg, Yield 58.67%).

LCMS (5%-95%, 1 cm, Rt=1.562 min): [M+H]+520.1

¹H NMR (400 MHZ, DMSO-d6) δ 8.42 (d, J=8.8 Hz, 1H), 7.80 (d, J=10.8 Hz, 1H), 7.31 (s, 1H), 5.60-5.54 (m, 1H), 5.42 (s, 2H), 5.25-5.06 (m, 2H), 3.16 (t, J=6.0 Hz, 2H), 2.40 (s, 3H), 2.12-2.11 (m, 2H), 2.07-2.05 (m, 3H), 1.93-1.79 (m, 2H), 0.91 (dd, J=6.4, 4.0 Hz, 6H), 0.86 (d, J=7.4 Hz, 3H).

### Example 9 Synthesis of Compound 9

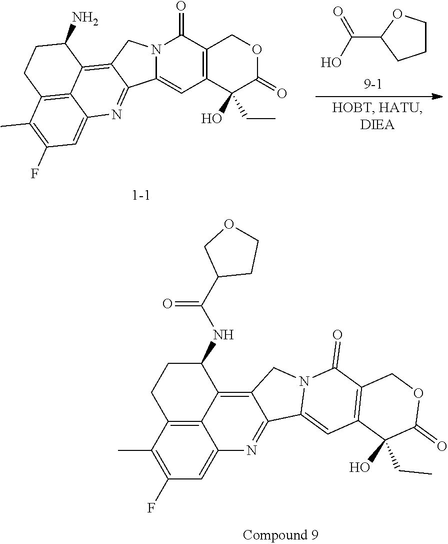 [SMILES: CC[C@@]1(O)C(=O)OCc2c1cc1n(c2=O)Cc2c-1nc1cc(F)c(C)c3c1c2[C@H](N)CC3 ; O=C(O)C1CCCO1 ; CC[C@@]1(O)C(=O)OCc2c1cc1n(c2=O)Cc2c-1nc1cc(F)c(C)c3c1c2[C@H](NC(=O)C1CCOC1)CC3]

To a solution of Compound 1-1 (50.0 mg, 0.11 mmol) in DMF (2.0 mL) was added Compound 9-1 (19.2 mg, 0.17 mmol), HOBT (22.3 mg, 0.17 mmol), HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% FA) (36%˜46%) to give Compound 9 (16.9 mg, Yield 30.65%).

LCMS (5%-95%, 1 cm, Rt=1.392 min): [M+H]+534.1

¹H NMR (400 MHZ, MeOD-d4) δ 7.54 (s, 1H), 7.52 (s, 1H), 5.64 (s, 1H), 5.53 (d, J=16.4 Hz, 1H), 5.33 (d, J=16.0 Hz, 1H), 5.21-5.08 (m, 1H), 4.96 (dd, J=18.8, 6.0 Hz, 1H), 4.06-3.87 (m, 3H), 3.85-3.77 (m, 1H), 3.25-3.13 (m, 2H), 3.12-3.04 (m, 1H), 2.38 (s, 3H), 2.35-2.30 (m, 1H), 2.29-2.14 (m, 3H), 1.95-1.90 (m, 2H), 0.98 (t, J=7.2 Hz, 3H).

### Example 10 Synthesis of Compound 10

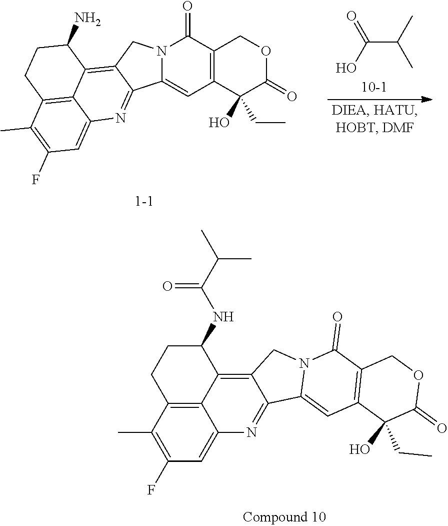

To a solution of Compound 1-1 (50.0 mg, 0.11 mmol) in DMF (1.0 mL) was added Compound 10-1 (10.9 mg, 0.12 mmol), HOBT (22.3 mg, 0.17 mmol) and HATU (62.7 mg, 0.17 mmol). The mixture was stirred at 0° C. for 10 min. DIEA (0.055 mL, 0.33 mmol) was added to the mixture and stirred at 20° C. for 16 hours. The mixture reaction was purified by C18 column chromatography eluted with ACN/H₂O (0.1% FA) (50%˜60%) to give Compound 10 (38.1 mg, Yield 68.33%).

LCMS (5%-95%, 1 cm, Rt=1.531 min): [M+H]+506.2.

¹H NMR (400 MHZ, DMSO-d6) δ 8.42 (d, J=8.7 Hz, 1H), 7.78 (d, J=11.0 Hz, 1H), 7.31 (s, 1H), 6.54 (s, 1H), 5.57-5.52 (m, 1H), 5.42 (s, 2H), 5.19-5.09 (m, 2H), 3.22-3.11 (m, 2H), 2.40 (s, 3H), 2.18-2.06 (m, 2H), 1.91-1.80 (m, 2H), 1.11 (t, J=7.3 Hz, 6H), 0.88 (t, J=7.3 Hz, 3H).

### Biological Examples

Exemplary compounds disclosed herein have been characterized in the following biological assays.

### Example 11 Cytotoxicity Test

Cytotoxicity of compounds was investigated using SHP77 (SCLC ATCC No. CRL-2195), NCI-H82 (SCLC, Jennio-bio No. JNO-22126A), NCI-H₅₂₄ (SCLC, Pricella NO. CL-0403), NCI-H226 (Lung cancer, Pricella NO. CL-0396), NCI-H₄₆₀ (LCLC, Pricella NO. CL-0299), OVCAR3 (Ovarian cancer, ATCC No. HTB-161), OV90 (Ovarian cancer, ATCC No. CRL-11732), LS1034 (Colon carcinoma, cobioer No. CBP60013), HT55 (Colon carcinoma, cobioer No. CBP60012), MDA-MB-468 (Breast cancer, ATCC No.htb-132) and NCI-N87 (Stomach cancer, ATCC No.CRL-5822). In brief, 5,000 cells/well of cancer cells were platted in white 96-well assay plates with flat clear bottom (Cat. No. BS-MP-96W, Biosharp) (excluding edge wells, which contained medium or PBS only) in 90 μL basic culture medium and were grown at 37° C. in a humidified incubator at 5% CO₂ atmosphere. After incubation overnight, each compound was added to the respective wells in an amount of 10 μl of 10× concentrate tests from 100 nM to 0.015 nM. After additional 72-hour incubation, plates were removed from incubator and equilibrated to room temperature. After approximately 30 mins, 50 μL of Cell counting-lite 2.0 Luminescent Cell Viability Assay (Cat. No. G7573, Promega) were added to each well. After shaking the plates at 450 rpm for 3 mins followed by 10-min incubation without shaking, luminescence was measured on the Envision plate reader (Equipment No. 2104, PerkinElmer) with integration time of 250 ms per well. Curves of luminescence versus concentration (nM) were fitted with GraphPad Prism Software. The results were showed in Table 1.

TABLE 1
IC₅₀ (nM) of exemplary compounds in different cell lines

| | Cell Line | | | | | | | | | | |
|---|---|---|---|---|---|---|---|---|---|---|---|
| Comp | | | | | | | | | | MDA- | |
| No. | SHP77 | H82 | H524 | H226 | H460 | OV90 | OVCAR3 | LS1034 | HT55 | MB-468 | NC-N87 |
| 1 | 45.7 | 96.0 | 4.4 | 4.8 | 10.2 | 3.9 | 92.9 | 63.5 | 129.7 | 2.5 | 6.2 |
| 2 | 301.2 | 154.5 | 40.4 | 21.0 | 15.5 | 15.2 | 208.1 | 205.1 | 314.9 | 10.8 | 13.0 |
| 3 | 60.0 | 50.2 | 11.3 | 3.6 | 8.9 | 5.1 | 91.2 | 41.7 | 68.1 | 2.1 | 5.3 |
| 4 | 655.1 | 1496.0 | 90.6 | 44.6 | 31.2 | 76.0 | 395.4 | 248.8 | 349.2 | 6.7 | 29.6 |
| 5 | 201.7 | 287.7 | 21.5 | 9.8 | 19.4 | 9.6 | 270.3 | 157.7 | 192.0 | 5.1 | 16.8 |
| 6 | 68.0 | 57.3 | 9.9 | 3.8 | 6.8 | 8.9 | 89.3 | 55.3 | 215.1 | 2.5 | 3.5 |
| 7 | 235.6 | 171.4 | 39.5 | 17.9 | 11.2 | 26.0 | 143.9 | 170.0 | 103.7 | 4.8 | 7.4 |
| 8 | 156.1 | 53.8 | 21.0 | 6.0 | 4.5 | 17.8 | 95.7 | 159.3 | 109.5 | 2.1 | 4.7 |
| 9 | 559.8 | 88.9 | 41.4 | 14.6 | 21.7 | 34.5 | 200.6 | 737.9 | 164.1 | 4.8 | 9.7 |
| 10 | 100.6 | 61.7 | 17.9 | 5.1 | 7.4 | 9.8 | 68.1 | 113.9 | 69.6 | 2.9 | 5.2 |

### Example 12 Membrane Permeability Test

Caco-2 cells purchased from ATCC were seeded onto 0.4 μm pore polycarbonate membranes (PC) in 96-well Corning Insert plates at 3.5×10⁴ cells/cm², and refreshed medium every 4˜5 days until to the 21st to 28th day for confluent cell monolayer formation.

The transport buffer in the study was HBSS with 10.0 mM HEPES at pH 7.40±0.05. Each test compound was tested at 2.00 μM in the presence or absence of 1.00 μM zosuquidar bi-directionally in duplicate. Digoxin was tested at 10.0 μM in the presence or absence of 1.00 μM zosuquidar bi-directionally in duplicate, while nadolol and metoprolol were tested at 2.00 μM in the absence of zosuquidar in A to B direction in duplicate. Final DMSO concentration was adjusted to less than 1%. The plate was incubated for 2 hours in CO₂ incubator at 37.0° C., with 5% CO₂ at saturated humidity without shaking. And all samples after mixed with acetonitrile containing internal standard were centrifuged at 3220×g for 10 min. Concentrations of test compounds in starting solution, donor solution and receiver solution were quantified by LC-MS/MS methodologies, using peak area ratio of analyte/internal standard. And permeation of lucifer yellow through the monolayer was measured to evaluate the cellular integrity.

The apparent permeability coefficient Papp (cm/s) was calculated using the equation:

$$Papp = ( dCr / dt ) \times Vr / ( A \times C 0 )$$

where dCr/dt is the cumulative concentration of compound in the receiver chamber as a function of time; Vr is the solution volume in the receiver chamber (0.0750 mL on the apical side, 0.250 mL on the basolateral side); A is the surface area for the transport, i.e., 0.143 cm2 for the area of the monolayer; C0 is the initial concentration in the donor chamber. The results were showed in Table 2.

TABLE 2
Membrane permeability of exemplary compounds

| Compound No. | A to B Mean Papp (10⁻⁶ cm/s) |
|---|---|
| 1 | 0.365 |
| 2 | 0.199 |
| 3 | 0.279 |
| 4 | 0.026 |
| 5 | 0.105 |
| 6 | 0.264 |
| 7 | 0.560 |
| 8 | 0.227 |
| 9 | 0.039 |
| 10 | 0.358 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What claimed is:

1. A compound selected from the group consisting of: 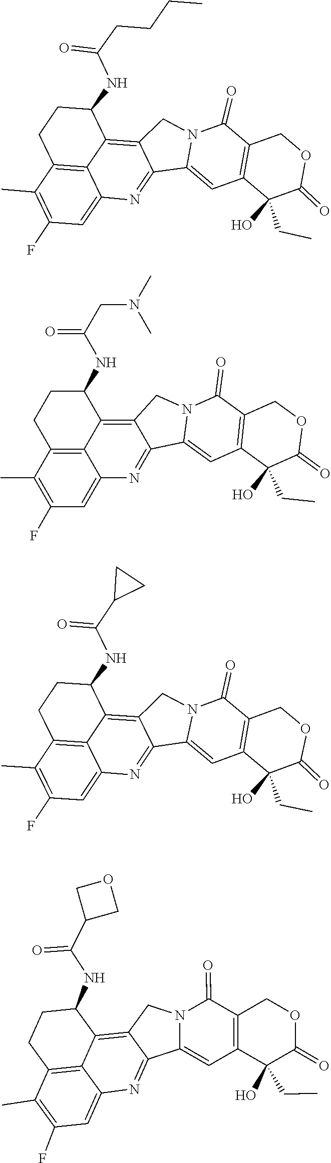 [SMILES: CCCCC(=O)N[C@@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@H](NC(=O)CN(C)C)CC3 ; CC[C@@]1(O)C(=O)OCc2c1cc1n(c2=O)Cc2c-1nc1cc(F)c(C)c3c1c2[C@H](NC(=O)C1CC1)CC3 ; CC[C@@]1(O)C(=O)OCc2c1cc1n(c2=O)Cc2c-1nc1cc(F)c(C)c3c1c2[C@H](NC(=O)C1COC1)CC3] 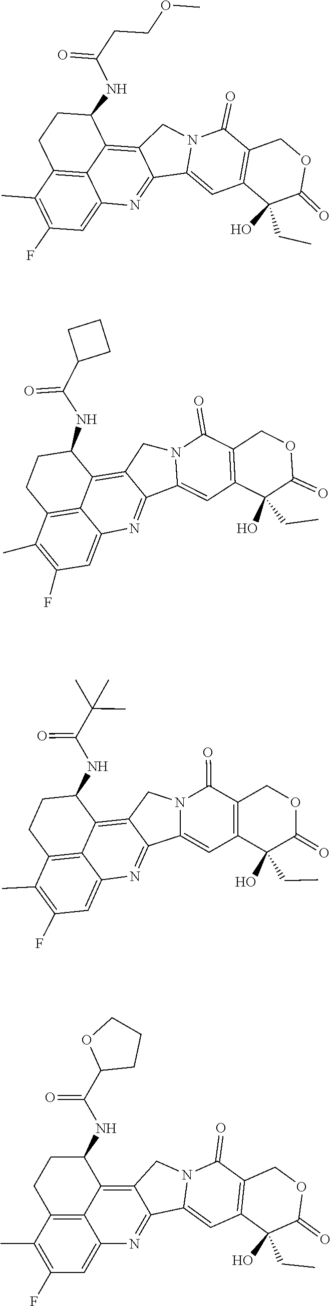 [SMILES: CC[C@@]1(O)C(=O)OCc2c1cc1n(c2=O)Cc2c-1nc1cc(F)c(C)c3c1c2[C@H](NC(=O)CCOC)CC3 ; CC[C@@]1(O)C(=O)OCc2c1cc1n(c2=O)Cc2c-1nc1cc(F)c(C)c3c1c2[C@H](NC(=O)C1CCC1)CC3 ; CC[C@@]1(O)C(=O)OCc2c1cc1n(c2=O)Cc2c-1nc1cc(F)c(C)c3c1c2[C@H](NC(=O)C(C)(C)C)CC3 ; CC[C@@]1(O)C(=O)OCc2c1cc1n(c2=O)Cc2c-1nc1cc(F)c(C)c3c1c2[C@H](NC(=O)C1CCCO1)CC3] 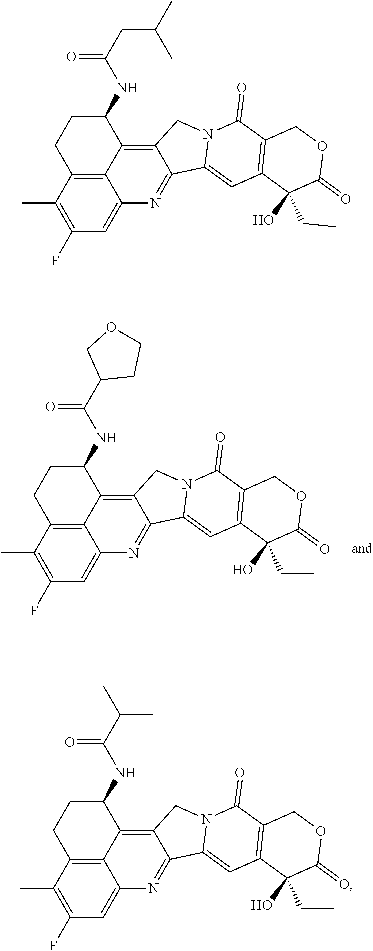 [SMILES: CC[C@@]1(O)C(=O)OCc2c1cc1n(c2=O)Cc2c-1nc1cc(F)c(C)c3c1c2[C@H](NC(=O)CC(C)C)CC3 ; CC[C@@]1(O)C(=O)OCc2c1cc1n(c2=O)Cc2c-1nc1cc(F)c(C)c3c1c2[C@H](NC(=O)C1CCOC1)CC3 ; CC[C@@]1(O)C(=O)OCc2c1cc1n(c2=O)Cc2c-1nc1cc(F)c(C)c3c1c2[C@H](NC(=O)C(C)C)CC3]
or a pharmaceutically acceptable salt thereof.

2. A pharmaceutical composition comprising the compound or a pharmaceutically acceptable salt thereof of claim 1, and a pharmaceutically acceptable excipient.

3. A method of treating cancer in a subject in need thereof, comprising administering to the subject an effective amount of the compound or a pharmaceutically acceptable salt thereof of claim 1, and/or the pharmaceutical composition of claim 2.

4. The method of claim 3, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

5. Use of the compound or a pharmaceutically acceptable salt thereof of claim 1 in the manufacture of a medicament for the treatment of cancer in a subject in need thereof.

6. A compound or a pharmaceutically acceptable salt thereof of claim 1, for use in the treatment of cancer in a subject in need thereof.
